(19) United States
(12) Patent Application Publication
(10) Pub. No.: US 2004/0092956 A1
(43) Pub. Date: May 13, 2004

(54) Catheter for removal of solids from surgical drains

(76) Inventors: John Liddicoat, Boston, MA (US); Alan Marc Gillinov, Orange Village, OH (US); Mark S. Goodin, Solon, OH (US)

Correspondence Address: PEARNE & GORDON LLP, 1801 EAST 9TH STREET, SUITE 1200, CLEVELAND, OH, 44114-3108, US

(21) Appl. No.: 10/415,843

(22) PCT Filed: Nov. 2, 2001
(86) PCT No.: PCT/US01/45648

(30) Foreign Application Priority Data
Nov. 3, 2000 (US) 60245796

(51) Int. Cl. A61B 17/22

(52) U.S. Cl. 606/127; 606/1

(57) ABSTRACT

A catheter is provided for clearing surgical drains of solid and semi-solid material that collects in and obstructs the drains. The catheter has a solids removal member at its distal end. Preferably, the solids removal member is an inflatable balloon, a deployable umbrella member, a deployable spring, a slicer, or a fixed spring. The solids removal member is effective to aid removal of collected solids from the surgical drain. An aspiration port is also provided at the distal end of the catheter to remove or aspirate collected solids that are dislodged from the surgical drain by the solids removal member. A method for using such a catheter is also provided.

### BACKGROUND OF THE INVENTION

1. Field of the Invention

This invention relates to a catheter for removing solid or semi-solid material from surgical drains. More particularly, it relates to a catheter assembly designed to clear surgical drains of clotted blood and particulate matter.

2. Description of Related Art

At the conclusion of many surgical procedures, drains are left in the patient to prevent accumulation of blood and other fluids. These drains are generally attached to a suction apparatus, facilitating removal of unwanted fluids. When drains are used to remove blood, these drains have a propensity to become occluded by blood clots, and the drains cease to function.

Clotting of chest drains is a particular problem in cardiac surgery. Because cardiac surgical patients receive large doses of anticoagulants during surgery and develop platelet dysfunction, most or all such patients bleed several hundred milliliters in the first 24 postoperative hours. In order to evacuate this blood, patients receive 2 to 4 chest tubes. Blood tends to clot in the chest tubes, and nurses attempt to ‘strip’ the drains to ensure their continued function. Unfortunately, such efforts are frequently unsuccessful. When a patient's chest tubes cease to function by becoming clogged or obstructed by clotted blood or other particulate matter, clotted blood may collect around the heart creating the life-threatening condition of cardiac tamponade. Up to 5% of cardiac surgical patients develop this important complication.

There is a need in the art for a device that can be used to clear surgical drains of clotted blood and thereby maintain their function. Such a device would be of great use, particularly in chest tubes for cardiac surgical patients. Preferably, such a device would function as an integral part of a surgical drain assembly (such as a chest tube assembly) in order to maintain a closed system and ensure sterility. Preferably, such a device could also be used to clear other types of surgical drains, including, e.g. biliary catheters and empyema tubes, of unwanted and compromising solid debris.

### SUMMARY OF THE INVENTION

A catheter for removing solid or semi-solid material from a surgical drain is provided. The catheter has a proximal region and a distal region, and a solids removal member disposed within its distal region. The distal region is suitable for insertion into a surgical drain to deliver the solids removal member within the surgical drain near the distal end of the drain.

A catheter assembly is also provided for removing solid or semi-solid material from a surgical drain. The assembly has a catheter having a proximal region and a distal region, a flexible protective sleeve having a first end and a second end, an aspiration port, and an aspiration vacuum port. The first end of the protective sleeve is attached to the catheter in the proximal region thereof. The sleeve is adapted to permit the distal region of the catheter to be extended into and withdrawn from the surgical drain. The aspiration port is located in the distal region of the catheter. The aspiration vacuum port is located in the proximal region of the catheter. The aspiration port and aspiration vacuum port are connected via an aspiration conduit. The distal region of the catheter is suitable for insertion into a surgical drain.

A method of removing solid or semi-solid material from a surgical drain is also provided. The method has the following steps: a) providing a catheter comprising a proximal region and a distal region, and that has a solids removal member and an aspiration port disposed in its distal region; b) applying a vacuum to the aspiration port; c) slowly inserting the catheter into the surgical drain so that the distal region of the catheter approaches the distal end of the surgical drain; d) actuating the solids removal member; and e) slowly withdrawing the catheter from the surgical drain.

### BRIEF DESCRIPTION OF THE DRAWINGS

FIG. 1 is a side view of a catheterized surgical drain assembly having a catheter according to the invention.

FIG. 2 is a side view of the invented catheterized surgical drain assembly with the catheter in a retracted position.

FIG. 3 is a side view as in FIG. 2, except that the catheter is in an extended position.

FIG. 4 is a perspective view of an invented catheter having an inflatable balloon according to a first preferred embodiment of the invention.

FIG. 5 is a cross-sectional view taken along line 5-5 of FIG. 4.

FIG. 6 is a side view, partially in section, of a catheterized surgical drain assembly having the invented catheter of FIG. 4, shown prior to the inflatable balloon penetrating a blood clot in the surgical drain.

FIG. 7 is a side view as in FIG. 6, shown after penetration of the blood clot by the inflatable balloon.

FIG. 8 is a schematic view of the distal region of a surgical drain with an invented catheter having a deployable umbrella member according to a second preferred embodiment of the invention.

FIG. 9 is a schematic view of the distal region of a surgical drain with an invented catheter having a deployable spring according to a third preferred embodiment of the invention.

FIG. 10 is a schematic view of the distal region of a surgical drain with an invented catheter having a slicer according to a fourth preferred embodiment of the invention.

FIG. 11 is a cross-sectional view of the slicer taken along line 11-11 in FIG. 10.

FIG. 12 is a schematic view of the distal region of a surgical drain with an invented catheter having a fixed spring according to a fifth preferred embodiment of the invention.

### DETAILED DESCRIPTION OF THE PREFERRED EMBODIMENTS OF THE INVENTION

In the description that follows, when a range such as 5 to 25 (or 5-25) is given, this means preferably at least 5 and, separately and independently, preferably not more than 25.

As used herein, the terms proximal and distal are generally construed with reference to a patient that has been fitted with a surgical drain. For example, the distal end of a surgical drain (or distal region of a catheter) is that end (or region) which is nearer or adjacent to the patient. Conversely, the proximal end of a surgical drain (or proximal region of a catheter) is that end (or region) which is further from the patient. Likewise, a distal element (or the distal side of an element) is nearer to the patient than a proximal element (or the proximal side of an element).

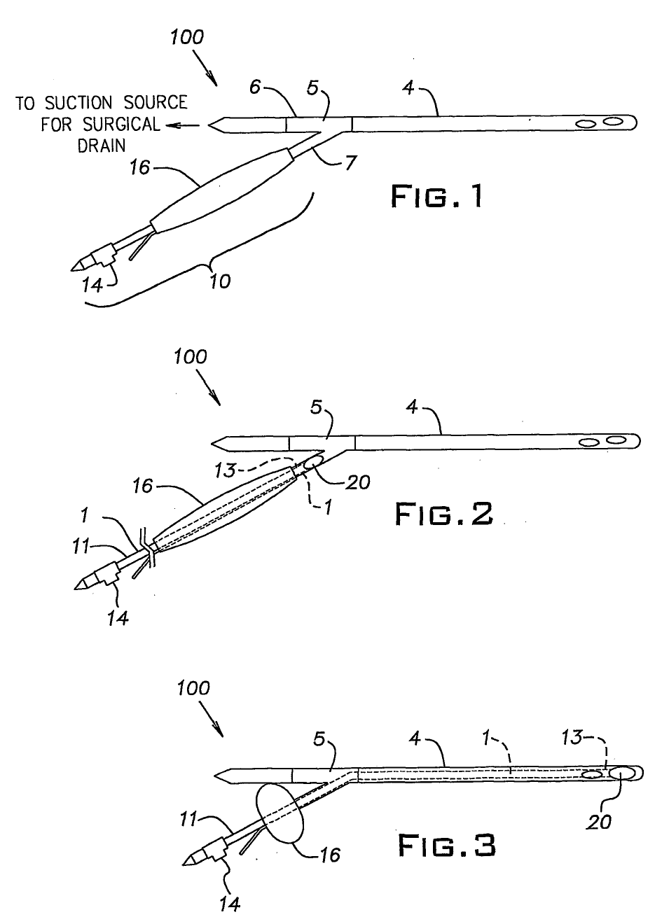

FIG. 1 shows a catheterized surgical drain assembly 100 according to the invention. The assembly 100 has a surgical drain 4 (such as a chest tube, biliary catheter, empyema tube, or other surgical drain), and a catheter assembly 10. The catheter assembly 10 is coupled to the surgical drain 4 via a catheter adaptor 5 having a suction lumen 6 and a catheter lumen 7. Preferably, the adaptor 5 has a one-way valve or check valve to prevent air or solid matter from proceeding through the adaptor 5 and into the drain 4 toward a patient. The adaptor 5 is preferably a y-adaptor as shown in FIG. 1, less preferably a tee-adaptor, less preferably some other configuration known in the art. A y-adaptor is preferred because it guides the insertion of catheter 1 into the drain 2 when the catheter 1 is in the extended position (explained below), without obstructing the suction path between the surgical drain 4 and the suction lumen 7 when in the retracted position (explained below).

The catheter assembly 10 has a catheter 1, a protective sleeve 16, and a solids removal member 20. In a preferred embodiment, the catheter assembly 10 is provided as an integrated catheter assembly with all the above components pre-assembled. In another preferred embodiment, the catheter assembly 10 is provided together with the catheter adaptor 5 and surgical drain 4 as an integrated catheterized surgical drain assembly. Preferably, the protective sleeve 16 is in the form of a flexible or expandable or elastic sheath having first and second ends. Preferably, the first end of the sheath is connected to the catheter 1 in proximal region 11, and its second end connected to the adaptor 5 (catheter lumen 7). The protective sleeve 16 provides a closed system for the catheterized surgical drain assembly 100, thereby ensuring a sterile environment within the assembly as the catheter is shifted between its retracted and extended positions as explained in the next paragraph. The protective sleeve 16 allows the catheter 1 to be reused without compromising the sterility of the system, and also preferably prevents exposure to secreted bodily fluids by healthcare personnel. Protective sleeve 16 is made from a flexible material, preferably a plastic or rubber material, e.g. latex, less preferably polypropylene or polyethylene, less preferably polytetrafluorgethylene, less preferably neoprene rubber, silicone or silicone rubber, less preferably ethylene propylene diene monomer (EPDM), less preferably any other suitable flexible material. Optionally, the protective sleeve 16 is provided in an accordion pattern such that it neatly expands and contracts as the catheter 1 is extended and retracted into/from the drain 4. The catheter 1 is made as customarily known in the art, from known or conventional materials.

Referring to FIGS. 2 and 3, an invented catheterized surgical drain assembly is shown with the catheter 1 in a retracted position and in an extended position respectively. As seen in FIG. 2, when the catheter 1 is in a retracted position, the catheter is retracted into the catheter lumen 7 of the adaptor 5, and does not obstruct the passageway of the surgical drain 4, or the path from the surgical drain 4 to the suction lumen 6. As seen in FIG. 3, when in the extended position, the catheter 1 extends through the catheter adaptor 5 (preferably a y-adaptor) into the surgical drain 4.

The catheter 1 has a proximal region 11 and a distal region 13. As can be seen in FIG. 4, the invented catheter 1 preferably has in its distal region 13 a solids removal member 20,and an aspiration port 8. The solids removal member 20 is advanced into the bore of the surgical drain 4 when the catheter 1 is in its extended position. The aspiration port 8 is preferably connected via an aspiration conduit 32 to an aspiration vacuum port 14 in the proximal region 11 of the catheter 1. The vacuum port 14 can be capped or sealed when not in use to maintain sterility and vacuum in the surgical drain 4.

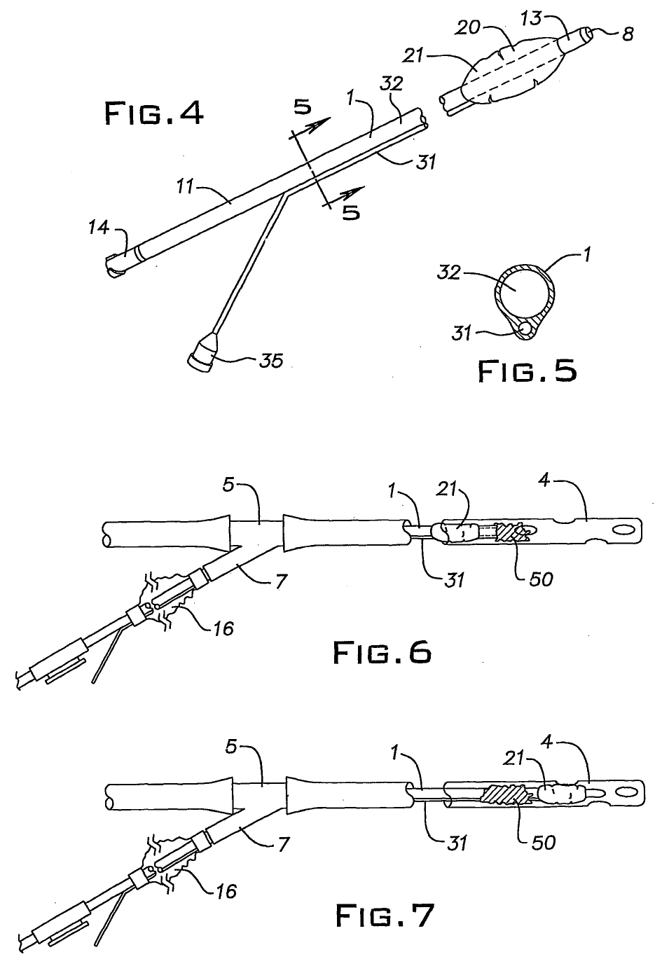

The catheter can be used to clear a surgical drain, such as a chest tube, of solid, semi-solid and liquid material by two mechanisms. Once the catheter is fully extended into the drain 4, suction may be used to aspirate material into the hollow bore or aspiration conduit 32 of the catheter 1. Intermittent occlusion of the aspiration vacuum port 14 in the proximal region 11 of the catheter causes intermittent suction at its distal aspiration port 8. In addition, actuation of the solids removal member 20 followed by withdrawal of the catheter 1 will clear the surgical drain 4 of larger collections of material and material adherent to the sides of the surgical drain 4. Aspiration is preferably achieved by connecting a suction source (preferably separate from that for the surgical drain 4) to the vacuum port 14 to aspirate solid, semi-solid and/or particulate material out of the surgical drain 4 through the aspiration port 8 of the catheter. The solids removal member 20 is actuated by an appropriate actuation means (as described herein or known in the art) that can be provided via an actuation conduit 31 between an actuation port 35 in the proximal region 11 of the catheter 1, and the solids removal member 20. Preferably, the actuation conduit 31 is separate from the aspiration conduit 32. (See FIG. 5). The catheter 1 may be used to clear the surgical drain 4 of debris by employing suction, withdrawal by the solids removal member 20, or both. In each of the following preferred embodiments, the catheter 1 can have (though does not require) an aspiration port 8 for aspiration of solid matter.

Referring to FIG. 4, the solids removal member 20 is an inflatable balloon 21 according to a first preferred embodiment of the invention. In this embodiment, the inflatable balloon 21 is inflated once the catheter 1 is in the fully extended position. Then the catheter 1 is withdrawn from the drain 4, removing blood clots and particulate matter too large to be aspirated into aspiration port 8, or that were stuck to the inner wall of the surgical drain 4. The balloon 21 is inflated by an appropriate inflation fluid, preferably air or saline, that is preferably injected into the actuation port 35 and delivered to the balloon 21 via the actuation conduit 31. Preferably, actuation port 35 is adapted to mate with a standard syringe for ease of balloon inflation.

A catheter 1 equipped with an inflatable balloon 21 as described is typically used in the following manner. (Though the following method is provided with reference to an inflatable balloon 21, it will be understood that the method is generally applicable to an invented catheter having a solids removal member other than an inflatable balloon). Once a caregiver or healthcare professional notes an apparent obstruction in a surgical drainage tube (i.e. indicated by cessation of movement of fluid through the drain 4, or collapsing of the tubing connecting the drain 4 to the collection unit), the caregiver first ensures the balloon 21 is uninflated (i.e. is in a collapsed position). If the catheter 1 is equipped with an aspiration port 8, the vacuum port 14 is sterilely attached to a suction source. In this manner, a continuous vacuum is applied to the aspiration port 8 to assist in eliminating thrombi 50 and particulate from the surgical drain 4. Continuous aspiration may aid in tunneling through a clot or thrombus 50 in order for the catheter 1 to penetrate the thrombus 50 and deliver the inflatable balloon 21 to the distal side of the thrombus So. (See FIGS. 6-7).

Once the vacuum has been applied, the catheter 1 is slowly inserted into the surgical drain 4 until it approaches the distal end of the drain 4. Next, the balloon 21 is actuated or inflated such that the balloon 21 engages and pushes against the inner wall surface of the drain 4. Once the balloon is inflated, the catheter 1 (and thereby inflated balloon 21) is slowly pulled back or withdrawn to the proximal end of the drain 4, with the inflated balloon 21 dislodging and pulling any particulate or thrombi 50 with it. Once such solids are dislodged from the inner wall of the drain 4, the solids are evacuated from the drain, either through the aspiration port 8 in the catheter 1, or through the suction lumen 6 by the surgical drain suction source. Following the above procedure, the balloon is de-actuated (i.e. deflated to a collapsed position) and the catheter 1 is withdrawn back into a retracted position within the catheter lumen 7. Once the catheter 1 is fully retracted, normal operation of the surgical drain 4 is resumed.

The above procedure could be performed at regular intervals (i.e. every 1, 2, 3, 4, 5, 6, 7, 8, 9, 10, 12, 24, 36, or 72, hours), or upon discovery of an occluded surgical drain 4. For surgical drains having very small diameters (i.e. 1-5, 1-3, or 1-2, mm) such as those employed for minimally invasive surgeries, the drain 4 is preferably cleaned via the above procedure at shorter intervals, preferably every 0.5-24, preferably 0.5-12, preferably 0.5-6, preferably 0.6-4, preferably 0.8-2, preferably about 1, hour(s).

Referring to FIG. 8, the solids removal member 20 is a deployable umbrella member 22 according to a second preferred embodiment of the invention. In this embodiment, the distal region 13 of the catheter 1 has a remotely deployable umbrella member 22. The catheter 1 is introduced into the surgical drain 4 with the umbrella member 22 in a collapsed position. Once the catheter 1 is fully extended with its tip near the distal end of the surgical drain 4, the umbrella member 22 is deployed into an open position as shown in FIG. 8. A preferred means of deploying the umbrella member is a guide wire 19 as known in the art. Guide wire 19 is attached at one end to the umbrella member 22 as shown in FIG. 8. The guide wire is threaded through the actuation conduit 31 to exit the actuation port 35 in the proximal region of the catheter where it can be manipulated by a caregiver to actuate the umbrella member 22. Less preferably, umbrella member 22 can be actuated via other known or conventional means.

When in the open position, the terminal edge 22a of umbrella member 22 preferably engages the inner wall of the drain 4. Similarly as described above with respect to the first preferred embodiment, the catheter 1 (and umbrella member 22) is slowly withdrawn from the drain 4, with the umbrella member 22 dislodging and pulling occluding thrombi 50 and particulate matter from the drain 4. Also, as in the first preferred embodiment, an aspiration port 8 can be provided, and continuous suction applied to aid clearing of solid or particulate matter from the surgical drain 4, and delivery of the umbrella member 22 to the distal side of any present thrombi 50.

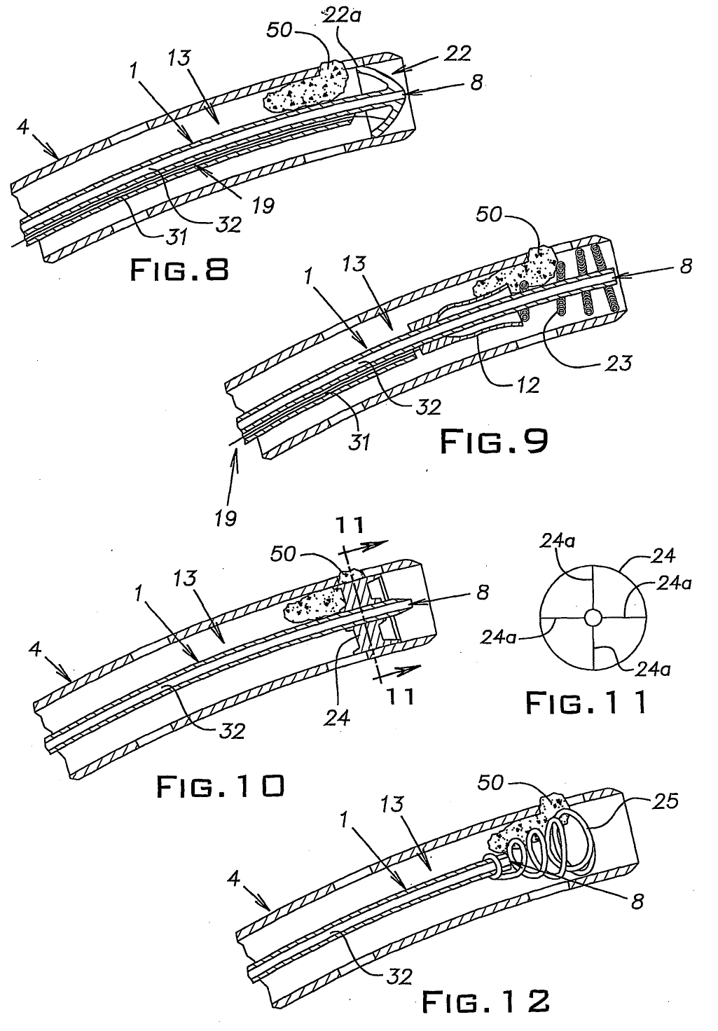

Referring to FIG. 9, the solids removal member 20 is a deployable spring 23 according to a third preferred embodiment of the invention. This embodiment is used in a similar manner to the deployable umbrella member embodiment previously described. The distal region 13 of the catheter 1 has a remotely deployable coiled spring 23. The catheter 1 is introduced into the surgical drain 4 with the spring 23 in a collapsed position enclosed in sheath 12. The sheath 12 is slidably engaged to the outer surface of the catheter 1, and causes the spring to collapse inward when the spring is pulled within the sheath 12. Once the catheter has been fully extended as previously described, the sheath 12 is actuated (i.e. retracted) causing the coiled spring 23 to deploy as shown in FIG. 9. When deployed, spring 23 preferably engages the inner surface of the surgical drain 4.

Preferably, the sheath 12 is actuated by a guide wire 19 similarly as described above. The spring 23 is captured within the sheath 12 by pushing the guide wire 19 through the actuation conduit 31 while holding the catheter 1 in place to prevent the advance of the spring 23. Conversely, the spring 23 is deployed by pulling the guide wire 19 in the direction of the proximal region 11 and away from the distal region 13 while holding the catheter 1 in place. With the coiled spring 23 deployed, the caregiver or healthcare professional removes thrombi 50 and particulate by slowly withdrawing the catheter 1, in the manner previously described.

Referring to FIG. 10, the solids removal member 20 is a slicer 24 according to a fourth preferred embodiment of the invention. The slicer 24 preferably has a circular cross-section as shown in FIG. 11, with both proximal and distal cutting edges. Preferably, the slicer 24 has a plurality of radial slats 24a as shown in FIG. 11, preferably at least 2 slats, more preferably 4 slats, each slat also having proximal and distal cutting edges. The slicer 24 is fixedly engaged to the exterior surface of the catheter 1. The slicer 24 has a slightly smaller diameter than the inner diameter of the surgical drain 4. Such slightly smaller diameter allows the slicer 24 to clear thrombi 50 and other debris while allowing translation of the slicer 24 along the drain 4. The slicer functions by inserting the catheter 1 into the surgical drain 4 to deliver the slicer to the distal end of the drain 4. Next, the catheter 1 is slowly withdrawn through the drain 4 such that the cutting edges of the slicer 24 cut up and dislodge entrained thrombi and particulate to be evacuated as previously described.

Referring to FIG. 12, the solids removal member 20 is a fixed spring 25 attached in the distal region 13 of the catheter 1 according to a fifth preferred embodiment of the invention. Preferably, the fixed spring 25 has a helical pattern, e.g. as shown in FIG. 12. The fixed spring 25 can be a coiled spring, which is then subsequently coiled into a helical pattern, or it can be an uncoiled strip of material (preferably metal) that has been oriented in a helical pattern. The catheter 1 having such a fixed spring 25 can be inserted into a surgical drain as previously described, and preferably is rotated such that the fixed spring 25 contacts and breaks up entrained thrombi 50 or other particulate. Next, the catheter 1 is withdrawn from the surgical drain 4, and the dislodged solids are evacuated either through aspiration port 8 in the catheter 1, or through the suction lumen 6 by the surgical drain suction source.

Although the hereinabove described embodiments of the invention constitute the preferred embodiments, it should be understood that modifications can be made thereto without departing from the scope of the invention as set forth in the appended claims.

What is claimed is:

1. A device for removing solid or semi-solid material from a surgical drain, the device comprising a catheter having a proximal region and a distal region, and a solids removal member disposed in said distal region of said catheter, said distal region being suitable for insertion into a surgical drain to deliver said solids removal member within said surgical drain.

2. A device according to claim 1, further comprising a flexible protective sleeve having a first end and a second end, wherein said first end of said protective sleeve is attached to said catheter in said proximal region thereof, said sleeve being adapted to permit the distal region of said catheter to be extended into and withdrawn from said surgical drain.

3. A device according to claim 2, wherein said protective sleeve is effective to maintain a sterile environment within said surgical drain, and to prevent exposure to secreted bodily fluids by healthcare personnel.

4. A device according to claim 2, wherein said protective sleeve is made from plastic or rubber.

5. A device according to claim 1, further comprising an aspiration port located in the distal region of said catheter, and an aspiration vacuum port located in the proximal region of said catheter, wherein said aspiration port and said aspiration vacuum port are connected via an aspiration conduit.

6. A device according to claim 1, further comprising an actuation port in the proximal region thereof, wherein said solids removal member is actuatable by an actuation means via an actuation conduit between said actuation port and said solids removal member.

7. A device according to claim 2, further comprising a catheter adaptor, wherein said second end of said protective sleeve is attached to said catheter adaptor.

8. A device according to claim 7, wherein said catheter adaptor is adapted to guide insertion of said catheter into said surgical drain when said catheter is in an extended position.

9. A device according to claim 8, wherein said catheter adaptor is a y-adaptor having a catheter lumen and a suction lumen, said second end of said protective sleeve being connected to said catheter lumen of said adaptor, and wherein said suction lumen and said surgical drain define a suction pathway for said surgical drain.

10. A device according to claim 9, wherein said catheter does not obstruct said suction pathway when said catheter is in a retracted position.

11. A device according to claim 2, wherein said catheter, solids removal member, and protective sleeve are provided as an integrated catheter assembly.

12. A device according to claim 11, wherein said catheter assembly is provided together with a catheter adaptor and said surgical drain as an integrated catheterized surgical drain assembly.

13. A device according to claim 1, wherein said solids removal member is an inflatable balloon.

14. A device according to claim 13, further comprising an actuation conduit between an actuation port in the proximal region of said catheter and said balloon, wherein said balloon is inflatable via an inflation fluid that is delivered to said actuation port.

15. A device according to claim 14, wherein said actuation port is adapted to mate with a syringe.

16. A device according to claim 1, wherein said solids removal member is a deployable umbrella member.

17. A device according to claim 16, wherein said deployable umbrella member is actuated by a guide wire connected thereto.

18. A device according to claim 1, wherein said solids removal member is a deployable spring.

19. A device according to claim 18, further comprising a sheath which is slidably engaged to said catheter, wherein said sheath a) is adapted to enclose said deployable spring to retain said spring in a collapsed position thereof, and b) is retractable from said spring via an actuation means causing said spring to deploy.

20. A device according to claim 19, wherein said actuation means is a guide wire.

21. A device according to claim 1, wherein said solids removal member is a slicer having proximal and distal cutting edges.

22. A device according to claim 21, wherein said slicer has a plurality of slats, each said slat having proximal and distal cutting edges.

23. A device according to claim 1, wherein said solids removal member is a fixed spring.

24. A device according to claim 23, wherein said fixed spring is an uncoiled strip of material oriented in a helical pattern.

25. A device according to claim 23, wherein said fixed spring is a coiled spring.

26. A method of removing solid or semi-solid material from a surgical drain comprising the steps of:
a) providing a catheter comprising a proximal region and a distal region, and having a solids removal member and an aspiration port disposed in the distal region thereof;
b) applying a vacuum to said aspiration port;
c) slowly inserting said catheter into said surgical drain so that the distal region of said catheter approaches a distal end of said surgical drain;
d) actuating said solids removal member; and
e) slowly withdrawing said catheter from said surgical drain.

27. A method according to claim 26, wherein during said withdrawal step (e), said actuated solids removal member is effective to dislodge and pull solid, semi-solid, particulate, or thrombi material from said surgical drain.

28. A method according to claim 26, wherein said solids removal member is an inflatable balloon.

29. A method according to claim 26, wherein said solids removal member is selected from the group consisting of a deployable umbrella, a deployable spring, a slicer, and a fixed spring.

30. A method according to claim 26, wherein said surgical drain is a chest tube.

31. A device for removing solid or semi-solid material from a surgical drain, the device comprising a catheter having a proximal region and a distal region, a flexible protective sleeve having a first end and a second end, an aspiration port, and an aspiration vacuum port, wherein said first end of said protective sleeve is attached to said catheter in said proximal region thereof, said sleeve being adapted to permit the distal region of said catheter to be extended into and withdrawn from said surgical drain, said aspiration port being located in the distal region of said catheter, said aspiration vacuum port being located in the proximal region of said catheter, wherein said aspiration port and said aspiration vacuum port are connected via an aspiration conduit, said distal region being suitable for insertion into a surgical drain.
